(19) United States
(12) Patent Application Publication
(10) Pub. No.: US 2006/0099643 A1
(43) Pub. Date: May 11, 2006

(54) Method for determining the regulation of xenobiotic removal

(76) Inventors: Roger F. Bone, Bridgewater, NJ (US); Dionisios Rentzeperis, Downingtown, PA (US)

Correspondence Address: WILSON SONSINI GOODRICH & ROSATI, 650 PAGE MILL ROAD, PALO ALTO, CA, 94304-1050, US

(21) Appl. No.: 10/522,396

(22) PCT Filed: Jul. 23, 2003
(86) PCT No.: PCT/US03/23241

§ 371 Date: Aug. 16, 2005

Related U.S. Application Data
(60) Provisional application No. 60/398,023, filed on Jul. 24, 2002.
(60) Provisional application No. 60/413,843, filed on Sep. 27, 2002.
(60) Provisional application No. 60/413,866, filed on Sep. 27, 2002.

(51) Int. Cl. G01N 33/53; G06F 19/00

(52) U.S. Cl. 435/7.1; 702/19

(57) ABSTRACT

The present invention relates generally to a method for identifying ligands that affect xenobiotic removal based upon their ability to modify the stability of receptors that regulate cytochrome P450 expression and/or drug transport proteins.

This application claims priority benefit to U.S. Provisional Application Nos. 60/398,023 filed Jul. 24, 2002, and 60/413,866 and 60/413,843, both filed Sep. 27, 2002, which are incorporated by reference herein in their entirety.

### FIELD OF THE INVENTION

The present invention relates generally to a method for identifyng ligands that affect xenobiotic removal based upon their ability to modify the stability of receptors that regulate cytochrome P450 expression and/or drug transport proteins.

### BACKGROUND OF THE INVENTION

Many drugs are metabolized by the cytochrome P450 family of oxido-reductases. The expression of these enzymes is regulated in part by receptors such as nuclear receptors.

Xenobiotics, such as drugs or steroid metabolites, can alter the expression of cytochrome P450 enzymes through interaction with the relevant receptor. For example, the pregnane X receptor (PXR or SXR) has been reported to stimulate the transcription of cytochrome P450 CYP3A4 monooxygenases and other genes involved in the detoxification and elimination of xenobiotics (Goodwin et al., Annu. Rev. Pharmacol. Toxicol. 42:1-23 (2002); the aryl hydrocarbon receptor (Ah or XRE) has been reported to activate the CYP1A and CYP1B family of P450's from inducers that are contained in cigarette smoking and barabacued food; the constitutive androstane receptor (CAR) activates the CYP2A and CYP2B family of P450's by xenobiotic inducers; and PPAR-alpha is reported to activate the CYP4A family of P450 by the prescribed class of the anti-lipidimid fibrates. See, e.g., Tredger et al., Hospital Pharmacist, 9, 167-173, (2002), and references cited within.

Drugs may also alter the expression of proteins that regulate drug clearance or efflux from the body. For example, it has been reported that the steroid and xenobiotic receptor (SXR), also known as the pregnane X receptor (SXR) regulates drug efflux by activating expression of the gene MDR1, which encodes the protein P-glycoprotein (ABCB1) (Schuetz & Strom, Nat Med. 7:536-537 and the references cited therein), Synold et at., Nat. Med. 7:584-590).

Thus, a drug can alter the expression of metabolizing enzymes that limit the activity of the drug or change the metabolism of other drugs, creating undesired and dangerous drug-drug interactions. Further, the effectiveness of xenobiotics can be limited by activation of the expression of a drug efflux pathway. It is currently difficult to predict effects on the level of expression of xenobiotic metabolizing enzymes or proteins effecting drug clearance due to species differences in gene regulation.

Nuclear receptors are members of a superfamily of transcription factors controlling cellular functions including reproduction, growth differentiation, lipid and sugar homeostasis. Their fimction is regulated by a diverse set of ligands (xenobiotics, hormones, lipids and other known and undiscovered ligands). To date 48 nuclear receptors have been identified, 28 with known ligands, the remaining ones classified as orphans. The biology of the receptors is complex and tissue specific (Shang & Brown, Science 295:2465-2468, (2002)) and the molecular mechanism of action appears to be a function of preferential recruitment of accessory proteins, referred as co-regulators, that modulate the function of these receptors in a ligand independent or dependent fashion. Recruitment of the appropriate co-regulator can result in gene transcription or repression.

A central theme in signal transduction and gene expression is the constitutive or inducible interaction of protein-protein modular domains. Knowledge of ligands that can potentiate these interactions will provide information on the nature of the molecular mechanisms underlying biological events and on the development of therapeutic approaches for the treatment of disease. Existing methods for the identification of ligands are cumbersome and limited particularly in the case of proteins of unknown function.

Panvera offers reagents for the discrimination of agonist from antagonist ligands for the estrogen receptor subtype beta and has presented publicly data on the preferential recruitment of co-activator proteins. See. e.g., Bolger et al., Environmental Health Perspectives 106:1-7 (1998); and Panvera corporate presentation presented at the Orphan Receptor Meeting San Diego, (June 2002). Their reagents are used in assays based on fluorescence resonance energy transfer (FRET).

There are publications on similar assays for other nuclear receptors (ER-α, the ERR and PPAR family) that are also based on FRET. See, e.g. Zhou et al., Molecular Endocrinology 12:1594-1604 (1998); and Coward et al., 98:8880-8884, (2001). Similar experiments have been done using Biacore technology. See, e.g., Cheskis et al., J. Biological Chemistry 11384-11391 (1997) and Wong et al.; Biochemistry 40:6756-6765 (2001).

Cellular assays exist where the readout is gene expression. See, e.g., Camp et al., Diabetes 49:539-547 (2001) and Kraichely et al., Endocrinology, 141:3534-3545, (2000). For example, Karo-Bio has developed a gene expression readout assay to include conformational sensitive peptide probes for discrimination of agonist from antagonist ligands for nuclear hormone receptors. See, e.g., Paige et al., PNAS 96:3999-4004 (1999) and Presentation by Karo-Bio at the Orphan Receptor Meeting, San Diego (June 2002).

Greenfield et al., Biochemistry 40:6446-6652 (2001) reports the thermal. stabilization of the ER-α receptor in the presence of estradiol. However, the reference does not teach the identification of a molecule as an agonist or an antagonist of the ER-α receptor.

The art discussed above suffers from several drawbacks. For example, in the analysis of nuclear receptors, gene expression readout assays and cell based assays, counter-screens are required to validate that ligands or co-regulators identified interact directly with the receptor of interest and not through other proteins that can produce a signal transduction or gene activation/repression assay readout. In addition, cell readout technology lacks the sensitivity in identifying weak ligands (typically compounds of affinities of greater than 1 μM are rarely identified), and is only applicable to compounds that have a good cell permeability profile. Other commercial in vitro assays require the knowledge of ligands for establishing competitive displacement assays, or the use of them as tools to validate FRET based co-regulator assays.

There remains a need for an accurate, reliable technology that facilitates the rapid, high-throughput identification of ligands for their effect on receptors that regulate the expression of drug metabolizing enzymes and the resultant effect of ligands on xenobiotic metabolism, as well as their effect on receptors that regulate the expression of drug efflux.

### SUMMARY OF THE INVENTION

In one embodiment, the present invention provides a method of determining the effect on a drug-metabolizing enzyme activity by a drug lead. The method comprises providing a drug lead that modifies the stability of a receptor regulating cytochrome P450 expression and screening the drug lead for its ability to further modify the stability of the receptor in the presence of one or more co-regulators. A further modification of the stability of the receptor in the presence of the drug lead and a co-regulator indicates whether the drug lead increases the activity of a drug-metabolizing enzyme.

In another embodiment, the method comprises providing a drug lead that shifts the thermal unfolding curve of a receptor regulating cytochrome P450 expression and screening the drug lead for its ability to further shift the thermal unfolding curve of the receptor in the presence of one or more co-regulators. A further shift in the thermal unfolding curve of the receptor in the presence of the drug lead and a co-regulator indicates whether the drug lead increases the activity of a drug-metabolizing enzyme.

In other embodiments, the method comprises screening a molecule for its ability to modify the stability of a receptor regulating cytochrome P450 expression and to further modify the stability of the receptor when in the presence of one or more co-activators. A molecule that modifies the stability of the receptor and further modifies the stability of the receptor when in the presence of a co-activator is identified as an agonist of xenobiotic metabolism.

In yet another embodiment, the method comprises screening a molecule for its ability to shift the thermal unfolding curve of a receptor regulating cytochrome P450 expression and to further shift the thermal unfolding curve of the receptor when in the presence of one or more co-activators. A molecule that shifts the thermal unfolding curve of the receptor and further shifts the thermal unfolding curve of the receptor when in the presence of a co-activator is identified as an agonist of xenobiotic metabolism.

In still another embodiment of the present invention, the method comprises screening a molecule for its ability to modify the stability of a receptor regulating cytochrome P450 expression. A molecule that does not modify the stability of the receptor is identified as a non-agonist of xenobiotic metabolism. Various embodiments of the present invention provide another method of identifyig a non-agonist of xenobiotic metabolism. The method comprises screening a molecule for its ability to shift the thermal unfolding curve of a receptor regulating cytochrome P450 expression. A molecule that does not shift the thermal unfolding curve of said receptor is identified as a non-agonist of xenobiotic metabolism.

Various embodiments of the present invention comprise (a) screening one or more of a multiplicity of molecules for their ability to modify the stability of a receptor regulating cytochrome P450 expression; wherein molecules that do not modify the stability of the receptor are identified as non-agonists of xenobiotic metabolism; and (b) screening molecules from step (a) that modify the stability of the receptor for their ability to further modify the stability of said receptor when in the presence of one or more co-repressors; wherein molecules that further modify the stability of the receptor when in the presence of a co-repressor are identified as non-agonists of xenobiotic metabolism.

Other embodiment of the present invention comprise (a) screening one or more of a multiplicity of molecules for their ability to shift the thermal unfolding curve of a receptor regulating cytochrome P450 expression; wherein molecules that do not shift the thermal unfolding curve of the receptor are identified as non-agonists of xenobiotic metabolism; and (b) screening molecules from step (a) that shift the thermal unfolding curve of the receptor for their ability to further shift the thermal unfolding curve of the receptor when in the presence of one or more co-repressors; wherein molecules that further shift the thermal unfolding curve of the receptor when in the presence of a co-repressor are identified as non-agonists of xenobiotic metabolism.

Some embodiments of the present invention provide methods of identifyg an agonist of drug clearance comprising screening a molecule for its ability to modify the stability of a receptor regulating expression of a drug transport protein and to further modify the stability of the receptor when in the presence of one or more co-activators; wherein a molecule that modifies the stability of the receptor and further modifies the stability of the receptor when in the presence of a co-activator is identified as an agonist of drug clearance.

Other embodiments of the present invention provide methods of identifying an agonist of drug clearance comprising screening a molecule for its ability to shift the thermal unfolding curve of a receptor regulating expression of a drug transport protein and to further shift the thermal unfolding curve of the receptor when in the presence of one or more co-activators; wherein a molecule that shifts the thermal unfolding curve of the receptor and further shifts the thermal unfolding curve of the receptor when in the presence of a co-activator is identified as an agonist of drug clearance.

Some embodiments of the present invention provide a methods of determining the effect on the activity of drug efflux of a drug lead comprising providing a drug lead that modifies the stability of a receptor regulating expression of a drug transport protein and screening the drug lead for its ability to further modify the stability of the receptor in the presence of one or more co-regulators; wherein a further modification of stability of the receptor in the presence of the drug lead and a co-regulator of said one or more co-regulators indicates whether the drug lead increases the activity of drug efflux.

Still other embodiments of the present invention provide methods of determining the effect on the activity of drug efflux of a drug lead comprising providing a drug lead that shifts the thermal unfolding curve of a receptor regulating expression of a drug transport protein and screening the drug lead for its ability to further shift the thermal unfolding curve of the receptor in the presence of one or more co-regulators; wherein a further shift in the thermal unfolding curve of the receptor in the presence of the drug lead and a co-regulator of said one or more co-regulators indicates whether the drug lead increases the activity of drug efflux.

Another embodiment of the present invention provides a method of determining the effect of a molecule on xenobiotic metabolism and/or drug clearance comprising screening a molecule for its ability to modify the stability of the SXR receptor and to further modify the stability of said receptor when in the presence of one or more co-activators; wherein a further modification of stability of the receptor in the presence of the molecule and a co-regulator of said one or more co-regulators indicates whether the molecule is an agonist or an antagonist of xenobiotic metabolism and/or drug clearance.

Yet another general embodiment of the present invention provides another method of determining the effect of a molecule on xenobiotic metabolism and/or drug clearance comprising screening a molecule for its ability to shift the thermal unfolding curve of the SXR receptor and to further shift the thermal unfolding curve of said receptor when in the presence of one or more co-regulators; wherein a further shift of the thermal unfolding curve of the receptor in the presence of the molecule and a co-regulator of said one or more co-regulators indicates whether the molecule is an agonist or an antagonist of xenobiotic metabolism and/or drug clearance.

Further features and advantages of the present invention are described in detail below with reference to the accompanying drawings.

### BRIEF DESCRIPTION OF THE DRAWINGS

FIG. 1 illustrates experimental results expected for the identification of an agonist ligand in the presence of a co-activator.

FIG. 2 illustrates experimental results expected for the identification of an antagonist ligand in the presence of a co-activator.

FIG. 3 illustrates the statistical probability of a ligand to induce an agonist response when interacting with SXR.

### DETAILED DESCRIPTION OF THE INVENTION

In the following description, reference will be made to various terms and methodologies known to those of skill in the biochemical and pharmacological arts. Publications and other materials setting forth such known terms and methodologies are incorporated herein by reference in their entireties as though set forth in full.

One advantage of the methods of the present invention is that neither gene expression readout and cell based assays, nor the use of known ligands to establish the assay are required. The ability to generate information in such a direct fashion allows the discovery of drugs with desired properties.

By use of isolated and/or purified proteins and peptides in a single unifying assay, one can identify ligands that are involved in modulating protein-protein interactions and predict biological response. Not only can ligands be identified, but also the intrinsic affinity for the target protein can be calculated which then can be used to correlate to biological activity.

Further, by the methods of the present invention, molecules such as drug candidates can be screened to determine whether they would alter the expression of metabolizing enzymes that limit the activity of the drug or change the metabolism of other drugs, creating undesired and dangerous drug-drug interactions; or how they affect mechanisms of drug clearance.

Data generated by methods of the present invention does not require counter-screening, as changes in the melting temperature of a receptor, such as a protein is a direct consequence of the thermodynamic linkae of the binding energy of macromolecules and ligands to the protein of interest. Further, affinities of a ligand to a receptor are more sensitive (affinities of pM to mM are determined) and the methods are not limited by compounds with poor cell permeability.

In embodiments of the present invention, methods are provided for the determination of the effects of molecules, such as drug candidates or leads, on drug metabolizing enzyme activity and xenobiotic metabolism based upon molecules that modify the stability of a receptor that regulates cytochrome P450 expression. Molecules, including drug candidates or leads, that modify the stability of the receptor can be screened in the presence of the receptor and one or more co-regulators for their ability to further modify the stability of the receptor. Whether the stability of the receptor is further modified is an indication as to whether the molecule is an agonist or an antagonist of the receptor when in the presence of the co-regulator. Based upon this information, the effect on a drug-metabolizing enzyme activity, and thus xenobiotic metabolism of a ligand can be determined.

In other embodiments of the invention, methods are provided for the determination of the effects of molecules, such as drug leads, on drug metabolizing enzyme activity and xenobiotic metabolism based upon the unfolding of a receptor that regulates cytochrome P450 expression due to a thermal change. Molecules that shift the thermal unfolding curve of the receptor can be screened in the presence of the receptor and one or more co-regulators for their ability to further shif the thermal unfolding curve of the receptor. Whether the thermal unfolding curve of the receptor is frrther shifted is an indication as to whether the molecule is an agonist or an antagonist of the receptor when in the presence of the co-regulator. Based upon this information, the effect on a drug-metabolizing enzyme activity, and thus xenobiotic metabolism of a ligand can be determined.

The term “receptor” encompasses peptides, proteins, nucleic acids, and other receptors. The term encompasses both enzymes and proteins which are not enzymes. The term encompasses monomeric and multimeric proteins. Multimeric proteins may be homomeric or heteromeric. The term encompasses nucleic acids comprising at least two nucleotides, such as oligonucleotides. Nucleic acids can be single-stranded, double-stranded or triple-stranded. The term encompasses a nucleic acid which is a synthetic oligonucleotide, a portion of a recombinant DNA molecule, or a portion of chromosomal DNA.

The term “receptor” also encompasses portions of peptides, proteins, and other receptors which are capable of acquiring secondary, tertiary, or quaternary structure through folding, coiling or twisting.

The receptor may be substituted with substituents including, but not limited to, cofactors, coenzymes, prosthetic groups, lipids, oligosaccharides, or phosphate groups.

More specifically, the receptors utilized in the present invention are co-regulator-dependent. By “co-regulator-dependent” it is meant that the receptor is capable of binding at least one ligand and binding at least one co-regulator. Further, the activity of the receptor, whether in a ligand dependent or independent function, is dependent upon, at least in part, by a co-regulator. Co-regulator dependent receptors include, but are not limited to, nuclear receptors.

Nuclear receptors, and the role of co-regulators relating thereto, are known in the art. See, e.g., Aranda and Pascual, Physiological Reviews 81:1269-1304 (2001); Collingwood et aL, Journal of Molecular Endocrinology 23:255-275 (1999); and Robyr et al., Molecular Endocrinology 23:329-347 (2000); and Lee et al., Cellular and Molecular Life Sciences 58:289-297 (2001). The references incorporated by reference herein in their entireties.

Further, the co-regulator dependent receptors encompass vertebrate species, including, but not limited to humans, as well as invertebrates, including but not limited to insects.

Illustratively, insects contain hundreds of nuclear receptors, for which ligands can be identified as agonists or antagonists. See Laudet, J. Molecular Endocrinology 19:207-226 (1997) and Maglich et al., Genome Biology 2:1-7 (2001) for a discussion of nuclear receptors present in vertebrates, nematodes and arthropods, the references incorporated by reference herein by their entireties.

More particularly, in embodiments of the invention, the term “receptor” refers to a receptor which regulates P450 expression, including, but not limited to, the steroid X receptor (SXR).

In other embodiments of the invention, the term “receptor” refers to a receptor which regulates drug transport protein expression, including, but not limited to, PXR and SXR.

The term “protein” encompasses full length or polypeptide fragments. The term “peptide” refers to protein fragments, synthetic or those derived from peptide libraries. As used herein, the terms “protein” and “polypeptide” are synonymous.

The term “co-regulator” refers to chemical compounds of any structure, including, but not limited to nucleic acids, such as DNA and RNA, and peptides that modulate the receptor in a ligand dependent or independent fashion. The term refers to natural, synthetic and virtual molecules. More specifically, the term refers to a peptide or polypeptide/protein, natural or synthetic that modulates the receptor in a ligand dependent or independent fashion. The term encompasses peptides that are derived from natural sequences or from phage display libraries. The peptide can be fragments of native proteins. More specifically, the term refers to co-activators and co-repressors, and even more specifically in embodiments of the invention to co-activators and co-repressors of a receptor regulating the expression of cytochrome P450 enzymes or drug transport proteins.

The term “co-activator” refers to a molecule which binds to a receptor and causes an activation of or an increase in an activity of the receptor. In embodiments of the invention, the term refers to molecules that bind to a receptor to induce gene transcription or to induce a signaling function (e.g. signal transduction).

The term “co-repressor” refers to a molecule which binds to a receptor and causes a deactivation or a decrease in an activity of the receptor. In embodiments of the invention, the term refers to molecules that bind to a receptor to repress gene transcription or to repress a signaling function (e.g. signal transduction).

The term “agonist” refers to a molecule which binds to a receptor and induces or recruits a co-activator for binding to the receptor. In embodiments of the invention, the term “agonist” refers to a molecule that binds to a nuclear receptor and recruits a co-activator. In these embodiments, the term more specifically refers to a molecule that alters gene expression by inducing conformational changes in a nuclear receptor that promote direct interactions with co-activators. In some embodiments, the agonist is a strong inducer of a co-activator dependent receptor. In other embodiments, the agonist is a partial agonist and a weak inducer of the co-regulator dependent receptor.

The term “antagonist” refers to a molecule which binds to a receptor and induces or recruits a co-repressor for binding to the receptor. In embodiments of the invention, the term “antagonist” refers to a molecule that binds to a nuclear receptor and recruits a co-repressor. In these embodiments, the term more specifically refers to a molecule that alters gene expression by inducing conformational changes in a nuclear receptor that promote direct interactions with co-repressors. In some embodiments, the antagonist is a non-inducer or non-agonist of a co-regulator dependent receptor.

The term “molecule” refers to a compound which is tested for binding to the receptor in the presence of or absence of additional compounds, such as co-regulators. This term encompasses chemical compounds of any structure, including, but not limited to nucleic acids, such as DNA and RNA, and peptides. The term refers to natural, synthetic and virtual molecules. The term includes compounds in a compound or a combinatorial library. The term also includes drug leads or drug candidates. The terms “molecule” and “ligand” are synonymous.

The terms “multiplicity of molecules,” “multiplicity of compounds,” or “multiplicity of containers” refer to at least two molecules, compounds, or containers.

A “thermal unfolding curve” is a plot of the physical change associated with the unfolding of a protein or a nucleic acid as a function of temperature.

The terms “bind” and “binding” refer to an interaction between two or more molecules. More specifically, the terms refer to an interaction, such as noncovalent bonding, between a ligand and a receptor, or a co-regulator and a receptor, or a ligand, receptor, and a co-regulator.

The term “modification of stability” refers to the change in the amount of pressure, the amount of heat, the concentration of detergent, or the concentration of denaturant that is required to cause a given degree of physical change in a target protein that is bound by one or more ligands, relative to the amount of pressure, the amount of heat, the concentration of detergent, or the concentration of denaturant that is required to cause the same degree of physical change in the target protein in the absence of any ligand. Modification of stability can be exhibited as an increase or a decrease in stability. Modification of the stability of a receptor by a ligand indicates that the ligand binds to the receptor.

The term “further modification of stability” refers to an additional modification of stability of the receptor when in the presence of a molecule known to modify the stability of the receptor and one or more additional molecules. More specifically, the one or more additional molecules can be co-regulators.

The term “unfolding” refers to the loss of structure, such as crystalline ordering of amino acid side-chains, secondary, tertiary, or quaternary protein structure. A receptor, such as a protein, can be caused to unfold by treatment with a denaturing agent (such as urea, guanidinium hydrochloride, or guanidinium thiosuccicinate), a detergent, by treating the receptor with pressure, by heating the receptor, or by any other suitable change.

The term “physical change” encompasses the release of energy in the form of light or heat, the absorption of energy in the form or light or heat, changes in turbidity and changes in the polar properties of light. Specifically, the term refers to changes measured by spectroscopy including infrared spectroscopy, fluorescent emission, fluorescent energy transfer, absorption of ultraviolet or visible light, changes in the polarization properties of light, changes in the polarization properties of fluorescent emission, changes in the rate of change of fluorescence over time (i.e., fluorescence lifetime), changes in fluorescence anisotropy, changes in fluorescence resonance energy transfer, changes in turbidity, and changes in enzyme activity. Preferably, the term refers to fluorescence, and more preferably to fluorescence emission. Fluorescence emission can be intrinsic to a protein or can be due to a fluorescence reporter molecule. The use of fluorescence techniques to monitor protein unfolding is well known to those of ordinary skill in the art. For example, see Eftink, M. R., Biophysical J. 66: 482-501 (1994).

An “unfolding curve” is a plot of the physical change associated with the unfolding of a protein as a function of parameters such as temperature, denaturant concentration, and pressure.

The term “modification of thermal stability” refers to the change in the amount of thermal energy that is required to cause a given degree of physical change in a target protein that is bound by one or more ligands, relative to the amount of thermal energy that is required to cause the same degree of physical change in the target protein in the absence of any ligand. Modification of thermal stability can be exhibited as an increase or a decrease in thermal stability. Modification of the thermal stability of a receptor by a ligand indicates that the ligand binds to the protein.

The term “shift in the thermal unfolding curve” refers to a shift in the thermal. unfolding curve for a receptor that is bound to a ligand, relative to the thermal unfolding curve of the protein in the absence of the ligand.

The term “further shift in the thermal unfolding curve” refers to an additional shift of the thermal unfolding curve of the receptor when in the presence of a molecule known to shift the thermal unfolding curve of the receptor and one or more additional molecules. More specifically, the one or more additional molecules can be co-regulators.

The term “contacting a receptor” refers broadly to placing the target protein in solution with the molecule to be screened for binding. Less broadly, contacting refers to the turning, swirling, shaking or vibrating of a solution of the receptor and the molecule to be screened for binding. More specifically, contacting refers to the micing of the receptor with the molecule to be tested for binding. Mixing can be accomplished, for example, by repeated uptake and discharge through a pipette tip. Preferably, contacting refers to the equilibration of binding between the target protein and the molecule to be tested for binding. Contacting can occur in the container or before the receptor and the molecule to be screened are placed in the container.

The term “container” refers to any vessel or chamber in which the receptor and molecule to be tested for binding can be placed. The term “container” encompasses reaction tubes (e.g., test tubes, microtubes, vials, cuvettes, etc.). In embodiments of the invention, the term “container” refers to a well in a multiwell microplate or microtiter plate.

In embodiments of the invention, molecules that bind to the receptor can be screened for their ability to bind to a receptor in the presence of one or more co-regulators. The term “screening” refers generally to the testing of molecules or compounds for their ability to bind to a receptor which is capable of denaturing or unfolding. The screening process can be a repetitive, or iterative, process, in which molecules are tested for binding to a protein in an unfolding assay.

In various embodiments of the present invention, the compounds are strong inducers of P450 expression. In other embodiments, the binding affinities of the strong inducers are about less than 5 μM, or about 4.5 μM, or about 4 μM, or about 3.5 μM, or about, 3 μM, or about 2.5 μM, or about 2 μM, or about 1.5 μM, or about 1 μM and the statistical probability of the agonist state of the strong inducer is about 0.8 to about 1.0. In yet another embodiment, the statistical probability of the strong inducer is at least about 0.8, or at least about 0.85, or at least about 0.9, or at least about 0.95, or at least about 1.0.

In other embodiments, the compounds are weak inducers of P450 expression. In other embodiments, the binding affinities of the weak inducers are about less than 5 μM, or about 4.5 μM, or about 4 μM, or about 3.5 μM, or about, 3 μM, or about 2.5 μM, or about 2 μM, or about 1.5 μM, or about 1 μM and the statistical probability of the agonist state of the weak inducer is about 0.4 to about 0.8. In another embodiment, the statistical probability of the weak inducer is between about 0.4 and 0.5, or between about 0.5 to about 0.6, or between about 0.6 to about 0.7, or between about 0.7 to about 0.8.

In another embodiment of the present invention, the compounds are non-inducers of P450 expression. In other embodiments, the binding affinities of the non- inducers are less than about 5 μM, or about 4.5 μM, or about 4 μM, or about 3.5 μM, or about, 3 μM, or about 2.5 μM, or about 2 μM, or about 1.5 μM, or about 1 μM and the statistical probability of the agonist state of the non-inducer is about 0.0 to about 0.4. In another embodiment, the statistical probability of the non-inducer less than about 0.05, or at less than about 0.1, or less than about 0.15, or less than about 0.2, or less than about 0.25, or less than about 0.3, or less than about 0.35, or less than about 0.4. In some embodiments, the weak inducer appears inactive.

In other embodiments, the compound is a weak inducer for P450 expression. In various embodiments, the weak inducer has a binding affnnity of greater than about 5 μM and a probability of an agonist state of about 0.4 to about 1.0, or between about 0.4 to about 0.5, or between about 0.5 to about 0.6, or between about 0.6 to about 0.7, or between about 0.7 to about 0.8, or between about 0.8 to about 0.9, or between about 0.9 to about 1.0.

In still another embodiment, the compound appears inactive for P450 expression. In various embodiments, the non-inducer has a binding affinity of greater than about 5 μM and a probability of an agonist state of less than about 0.4, or less than about 0.35, or less than about 0.3, or less than about 0.25, or less than about 0.2, or less than about 0.15, or less than about 0.1, or less than about 0.5.

As mentioned above, in accordance with various embodiments of the invention, the effect of a molecule, such as a drug lead, on a drug-metabolism enzyme activity and xenobiotic metabolism can be identified based upon modification of stability of a receptor regulating cytochrome P450 expression. Molecules that modify the stability of the receptor can be screened for their ability to further modify the stability of the receptor in the presence of one or more co-regulators.

In one embodiment, to perform the screening, one or molecules (e.g. of a set) that modify the stability of the receptor can be contacted with the receptor and one of more co-regulators in each of a multiplicity of containers. The receptor in each of the containers can then be treated to cause the target protein to unfold. A physical change associated with the unfolding of the receptor can be measured. An unfolding curve for the receptor for each of containers can then be generated. Each of the unfolding curves may be compared to (1) each of the other unfolding curves and/or to (2) the unfolding curve for the receptor in the absence of (i) any of the molecules from the set and/or (ii) the co-regulators.

Based upon the generated data, one can determine whether the screened molecules further modify the stability of the receptor in the presence of the co-regulators, and thus identify whether the molecules are agonists or antagonists of xenobiotic metabolism. A further modification of stability of the receptor is indicated by a further change in the unfolding curve of the receptor.

In other embodiments of the invention, the effect of a molecule, such as a drug lead, on a drug-metabolism enzyme activity and thus xenobiotic metabolism can be identified by analyzing molecules that modify the thermal stability, and more particularly, shift the thermal unfolding curve of a receptor that regulates cytochrome P450 expression. Molecules that shift the thermal unfolding curve of the receptor can be screened for their ability to further shift the thermal unfolding curve of the receptor in the presence of one or more co-regulators.

In an embodiment of the invention, the screening can be accomplished by contacting the receptor with one or more of ligands (e.g., of a set) that shift the thermal unfolding curve of the receptor with one or more co-regulators in each of a multiplicity of containers. The multiplicity of containers can be heated, and a physical change associated with the thermal unfolding curve for the receptor as a function of temperature can be measured for each of the containers. A thermal unfolding curve for the receptor as a function of temperature can then be generated. The thermal unfolding curves that are generated can be compared with (1) each of the other thermal unfolding curves and/or to (2) the thermal unfolding curve for the receptor in the absence of (i) any of the molecules from the set and/or (ii) the co-regulators.

In embodiments of the screening method, the containers can be heated in intervals, over a range of temperatures. The multiplicity of containers may be heated simultaneously. A physical change associated with the thermal unfolding of the receptor can be measured after each heating interval. In an alternate embodiment of this method, the containers can be heated in a continuous fashion.

In embodiments of the invention, in generating an unfolding curve for the receptor, a thermal unfolding curve can be plotted as a function of temperature for the receptor in each of the containers.

In an embodiment of the invention, comparing the thermal unfolding curves can be accomplished by comparing the midpoint temperatures, Tₘ of each unfolding curve. The “midpoint temperature, Tₘ” is the temperature midpoint of a thermal unfolding curve. The Tₘ can be readily determined using methods well known to those skilled in the art. See, e.g., Weber, P. C. et al., J. Am. Chem. Soc. 116:2717-2724 (1994); and Clegg, R. M. et al., Proc. Natl. Acad. Sci. U.S.A. 90:2994-2998 (1993).

For example, the Tₘ of each thermal unfolding curve can be identified and compared to the Tₘ obtained for (1) the other thermal unfolding curves and/or to (2) the thermal unfolding curve for the receptor in the absence of (i) any of the molecules from the set and/or (ii) the co-regulators in the containers.

Alternatively or additionally, an entire thermal unfolding curve can be similarly compared to other entire thermal unfolding curves using computer analytical tools. For example, each entire thermal unfolding curve can be compared to (1) the other thermal unfolding curves and/or to (2) the thermal unfolding curve for the receptor in the absence of (i) any of the molecules from the set and/or (ii) the co-regulators in the containers.

Based upon the generated data, one can determine whether any of the screened molecules further shift the thermal unfolding curve of the receptor in the presence of a co-regulator, and thus identify whether a molecule is an agonist or antagonist of xenobiotic metabolism.

The methods of the present invention that involve determing whether molecules that shift and/or further shift the thermal unfolding curve of a receptor are distinct from methods that do not involve determining whether molecules shift and/or further shift the thermal unfolding curve of a receptor, such as assays of susceptibility to proteolysis, surface binding by protein, antibody binding by protein, molecular chaperone binding of protein, differential binding to immobilized ligand, and protein aggregation. Such assays are well-known to those of ordinary skill in the art For example, see U.S. Pat. Nos. 5,585,277 and 5,679,582. These approaches disclosed in U.S. Pat. Nos. 5,585,277 and 5,679,582 involve comparing the extent of folding and/or unfolding of the protein in the presence and in the absence of a molecule being tested for binding. These approaches do not involve a determination of whether any of the molecules that bind to the receptor shift the thermal unfolding curve of the receptor.

As discussed above, molecules that modify the stability of the receptor can be screened for the ability to further modify the stability of the receptor in the presence of a co-regulator. For example, molecules that are known to modify the stability of the receptors can be screened against a panel of identified co-regulators for the receptor, including co-activators and/or co-repressors. For convenience, the molecules known to modify the stability of the receptor are referred to as a “set” of molecules.

If the stability of the receptor is further modified in the presence of a molecule from the set and a co-activator of the receptor as compared to the receptor and the molecule from the set alone, then this is an indication that the molecule from the set is an agonist of the receptor when in the presence of the co-activator. In this way, it can be determined that the molecule can increase the effect on a drug-metabolizing enzyme activity and/or otherwise be an agonist of xenobiotic metabolism.

If the stability of the receptor is further modified in the presence of a molecule from the set and a co-repressor of the receptor as compared to the receptor and the molecule from the set alone, then this is an indication that the molecule from the set is an antagonist of the receptor when in the presence of the co-repressor. In this way, it can be determined that the molecule can decrease the effect on a drug-metabolizing enzyme activity and/or otherwise be an antagonist of xenobiotic metabolism.

Similarly, molecules that shift the thermal unfolding curve of the receptor can be screened for the ability to further shift the thermal unfolding curve of the receptor in the presence of a co-regulator. For example, molecules that are known to shift the thermal unfolding curve of the receptor can be screened against a panel of identified co-regulators for the receptor, including co-activators and/or co-repressors. For convenience, the molecules that are known to shift the thermal unfolding curve of the receptor are referred to as a “set” of molecules.

If the thermal unfolding curve of the receptor is further shifted in the presence of a molecule from the set and a co-activator of the receptor as compared to the receptor and the molecule from the set alone, then this is an indication that the molecule from the set is an agonist of the receptor when in the presence of the co-activator. In this way, it can be determined that the molecule can increase the effect on a drug-metabolizing enzyme activity and/or otherwise be an agonist of xenobiotic metabolism.

If the thermal unfolding curve of the receptor is further shifted in the presence of a molecule from the set and a co-repressor of the receptor as compared to the receptor and the molecule from the set alone, then this is an indication that the molecule from the set is an antagonist of the receptor when in the presence of the co-repressor. In this way, it can be determined that the molecule can decrease the effect on a drug-metabolizing enzyme activity and/or otherwise be an antagonist of xenobiotic metabolism.

The present invention also provides methods for identifying agonists or antagonists of xenobiotic metabolism based on the lack of further modification of stability and/or a lack of further shift in the unfolding curve of a receptor regulating cytochrome P450 expression.

By “lack of further modification of stability of the receptor” or “no further modification of stability of the receptor,” it is meant that there is either an insignificant further change or no further change in the stability of the receptor in the presence of both a molecule from the set and a co-regulator (as compared to the receptor and the molecule from the set).

By “lack of further shift in the thermal unfolding curve of the receptor” or “no further shift in the thermal unfolding curve of the receptor,” it is meant that there is either an insignificant further change or no further change in the shift of the thermal unfolding curve of the receptor in the presence of a molecule from the set and of a co-regulator (as compared to the receptor and the molecule from the set).

In embodiments of the invention, an antagonist of xenobiotic metabolism can be identified based on the lack of flrter modification of stability and/or lack of further shift in the thermal unfolding curve of a receptor regulating cytochrome P450 expression when in the presence of a co-activator. In other embodiments of the invention, an agonist of xenobiotic metabolism can be identified based on the lack of further modification of stability and/or lack of further shift in the thermal unfolding curve of a receptor regulating cytochrome P450 expression when in the presence of a co-repressor.

An antagonist of xenobiotic metabolism can be identified by screening one or more of a set of molecules that modify the stability of the receptor for their ability to further modify the stability of the receptor in the presence of one or more co-activators. Methods for screening the molecules from the set for their effect on further modifying the stability of the receptor are described above. If there is no further modification of the stability of the receptor in the presence of a molecule of the set and a co-activator, then this is an indication that such molecule of the set is an antagonist of the receptor when in the presence of the co-activator. In this way, such molecule can be determined to be an antagonist of xenobiotic metabolism.

An antagonist can also be identified by screening one or more of a set of molecules that shift the thermal unfolding curve of the receptor for their ability to further shift the thermal unfolding curve of the receptor in the presence of one or more co-activators. Methods for screening one or more molecules of the set for their ability to further shift the thermal unfolding curve of the receptor are described above. If there is no further shift in the thermal unfolding curve of the receptor in the presence of a molecule of the set and a co-activator, then this is an indication that such molecule of the set is an antagonist of the receptor when in the presence of the co-activator, and thus can be determined to be an antagonist of xenobiotic metabolism.

An agonist of xenobiotic metabolism can be identified by screening one or more of a set of molecules that modify the stability of the receptor for their ability to further modify the stability of the receptor in the presence of one or more co-repressors. Methods for screening the molecules from the set for their effect on further modifying the stability of the receptor are described above. If there is no further modification of the stability of the receptor in the presence of a molecule of the set and a co-repressor, then this is an indication that such molecule of the set is an agonist of the receptor when in the presence of the co-repressor. In this way, such molecule is determined to be an agonist of xenobiotic metabolism.

An agonist can also be identified by screening one or more of a set of molecules that shift the thermal unfolding curve of the receptor for their ability to further shift the thermal unfolding curve of the receptor in the presence of one or more co-repressors. Methods for screening one or more molecules of the set for their ability to further shift the thermal unfolding curve of the receptor are described above. If there is no further shift in the thermal unfolding curve of the receptor in the presence of a molecule of the set and a co-repressor, then this is an indication that such molecule of the set is an agonist of the receptor when in the presence of the co-repressor, and thus an agonist of xenobiotic metabolism.

Other embodiments of the present invention include methods of identifying non-agonists of xenobiotic metabolism. By “non-agonist” it is meant that the molecule, such as a drug candidate or lead, is an antagonist for a receptor regulating cytochrome P-450 expression when in the presence of a co-regulator, or one that does not bind to the receptor at all, and therefore does not increase the expression of drug-metabolizing enzymes.

For example, a non-agonist of xenobiotic metabolism can be identified by screening a molecule for its ability to modify the stability of a receptor regulating cytochrome P450 expression. If the molecule does not modify the stability of the receptor, the molecule can be identified as a non-agonist of xenobiotic metabolism.

In another embodiment, a non-agonist of xenobiotic metabolism can be identified by screening a molecule for its ability to shift the thermal unfolding curve of a receptor regulating cytochrome P450 expression. If the molecule does not shift the thermal unfolding curve of the receptor, the molecule can be identified as a non-agonist of xenobiotic metabolism.

In yet another embodiment, non-agonists of xenobiotic metabolism can be identified by screening one or more of a multiplicity of molecules for their ability to modify the stability of a receptor regulating cytochrome P450 expression. Molecules that do not modify the stability of the receptor are identified as non-agonists of xenobiotic metabolism. Molecules that do modify the stability of the receptor can be screened for their ability to firther modify the stability of the receptor when in the presence of one or more co-repressors. Molecules that further modify the stability of the receptor when in the presence of a co-repressor can be identified as non-agonists of xenobiotic metabolism.

In still yet another embodiment, non-agonists of xenobiotic metabolism can be identified by screening one or more of a multiplicity of molecules for their ability to shift the thermal unfolding curve of a receptor regulating cytochrome P450 expression. Molecules that do not shift the thermal unfolding curve of the receptor are identified as non-agonists of xenobiotic metabolism. Molecules that do shift the thermal unfolding curve of the receptor can be screened for their ability to further shift the thermal unfolding curve of the receptor when in the presence of one or more co-repressors. Molecules that further shift the thermal unfolding curve of the receptor when in the presence of a co-repressor can be identified as non-agonists of xenobiotic metabolism.

Methods have been described above for the identification of agonists and antagonists of xenobiotic metabolism based on providing molecules that are known to modify the stability and/or shift the thermal unfolding curve of the receptor and screening such molecules for their ability to furter modify the stability of and/or shift the thermal unfolding curve of the receptor. The invention also encompasses methods for the providing of such molecules in conjunction with the identification of such molecules as agonists or antagonists xenobiotic metabolism. Such methods are particularly useful in identifying ligands for orphan receptors, for which ligands that bind to the receptor are not known.

Molecules that modify the stability and/or shift the thermal unfolding curve of the receptor (referred to above as a “set” for convenience) can be obtained by the screening of a multiplicity of different molecules. For example, molecules that modify the stability of the receptor can be obtained by the screening of one or more of a multiplicity of different molecules for their ability to modify the stability of the receptor. Similarly, molecules that shift the thermal unfolding curve of the receptor can be obtained by the screening of one or more of a multiplicity of different molecules for their ability to shift the thermal unfolding curve of the receptor. In embodiments of the invention, the number of molecules that can be screened range from about one thousand to one million.

Molecules can be screened for their ability to modify the stability of the receptor by a method similar to the screening method described above for identifying agonists or antagonists. For example, the receptor can be contacted with one or more of a multiplicity of different molecules in each of a multiplicity of containers. The receptor in each of the multiplicity of containers can be treated to cause it to unfold. A physical change associated with the unfolding of the receptor can be measured. An unfolding curve for the receptor for each of the containers can be generated. Each of these unfolding curves can be compared to (1) each of the other unfolding curves and/or to (2) the unfolding curve for the receptor in the absence of any of the multiplicity of different molecules.

Based upon the generated data, one can determine whether any of the screened molecules modify the stability of the receptor. A modification of stability of the receptor is indicated by a change in the unfolding curve of the receptor. If a molecule modifies the stability of the receptor, it can then be screened to identify whether it is an agonist or an antagonist of a receptor regulating cytochrome P450 expression when in the presence of a co-regulator by the methods described above.

Similarly, molecules can be screened for their ability to shift the thermal unfolding curve of the receptor by a method similar to the screening method for identifying agonists or antagonists. For example, the receptor can be contacted with one or more of a multiplicity of different molecules in each of a multiplicity of containers. The containers can be heated, and a physical change associated with the thermal unfolding of the receptor can be measured in each of the containers. A thermal unfolding curve for the receptor can be generated as a function of temperature for each of the containers.

The thermal unfolding curves can be compared with (1) each of the other thermal unfolding curves and/or to (2) the thermal unfolding curves for the receptor in the absence of any of the multiplicity of different molecules. In embodiments of the invention, the Tₘ of each thermal unfolding curve can be identified and compared to the Tₘ obtained for (1) the other thermal unfolding curves and/or to (2) the thermal unfolding curve for the receptor in the absence of any of the multiplicity of molecules. Alternatively, each entire thermal unfolding curve can be compared to (1) the other thermal unfolding curves and/or to (2) the thermal unfolding curve for the receptor in the absence of any of the multiplicity of different molecules.

Based upon the generated data, one can determine whether any of the screened molecules shift the thermal unfolding curve of the receptor. If a molecule shifts the thermal unfolding curve of the receptor, it can then be screened to identify whether it is an agonist or an antagonist of a receptor regulating cytochrome P450 expression when in the presence of a co-regulator by the methods described above.

In embodiments of the invention, a microplate thermal shift assay is a particularly useful means for identifying ligands and identing such ligands as agonists or antagonists of xenobiotic metabolism. The microplate thermal shift assay is a direct and quantitative technology for assaying the effect of one or more molecules on the thermal stability of a target receptor.

The theory, concepts, and application of the microplate thermal shift assay, and apparatuses useful for practicing the microplate thermal shift assay are described in U.S. Pat. Nos. 6,020,141; 6,036,920; 6,291,191; 6,268,218; 6,232,085; 6,268,158; 6,214,293; 6,291,192; and 6,303,322, which are all hereby incorporated by reference in their entireties. The microplate thermal shift assay discussed in these references can be used to implement the screening methods described above.

The microplate thermal shift assay provides a thermodynamic readout of ligand binding affinity. The assay depends upon the fact that each functionally active receptor is a highly organized structure that melts cooperatively at a temperature that is characteristic for each receptor and representative of its stabilization energy. When a molecule binds to a receptor, the receptor is stabilized by an amount proportional to the ligand binding affinity, thus shifting the midpoint temperature to a higher temperature.

There are many advantages to using the thermal shift assay since it does not require radioactively labeled compounds, nor fluorescent or other chromophobic labels to assist in monitoring binding. The assay takes advantage of thermal unfolding of biomolecules, a general physical chemical process intrinsic to many, if not all, drug target biomolecules. General applicability is an important aspect of this assay, as it obviates the necessity to invent a new assay every time a new therapeutic receptor protein becomes available.

Further, using the thermal shift assay, owing to the proportionality of the Tₘ and the ligand binding affinity, ligand binding affinities ranging from greater than 10 micromolar to less than 1 nanomolar can be measured in a single well experiment Thus, the thermal shift assay can be used to quantitatively detect ligand binding affinity to a receptor alone and/or in the presence of a co-regulator.

Further, the thermal shift assay can be used in the identification of agonists and antagonists on a quantitative basis based upon the change in the Tₘ between the ligand and receptor and the ligand, receptor and a co-regulator. The microplate thermal shift assay can be used to measure multiple ligand binding events on a single receptor as incremental or additive increases of the receptor's melting temperature.

The present invention has particular utility in the identification of ligands and the identification of such ligands as agonist or antagonist in nuclear receptors, such as a nuclear receptor that regulates cytochrome P450 expression.

For example, the present invention may be used to identify ligands that interact with the ligand binding domain of ER-α and ER-β, the two subtypes of the estrogen receptor family. These domains contain two known binding sites, one for estrogen like compounds and another for co-regulator proteins. The present invention can be used to identity ligands that interact with the estrogen receptor. These ligands produce an observed increase in the stability of the receptor which is proportional to the inherent affinity of the ligand.

The ligand binding domain of nuclear receptors, and co-regulator proteins can be expressed using standard recombinant methods in Escherichia coli. Co-regulator peptides can be synthesized using standard methods. The melting temperature of the purified protein of interest can be determined by the microplate thermal shift assay in the absence and in the presence of small molecule ligands.

Molecules are provided that stabilize the receptor of interest. Such small molecules can be obtained by screening in the microplate thermal shift assay, as referred to above. The number of small molecules in the screen can range from about one thousand to one million. The small molecules can be natural or synthetic.

Once a set of small molecules have been identified to stabilize the protein of interest, then these molecules can be screened against a panel of co-regulators, such as proteins or peptide fragments, to measure their effect on the thermal stability of the protein. If a synergistic effect is observed, the compounds can be classified as agonist or antagonist. Equilibrium constants are calculated for both ligand and co-regulator and related to biological responses.

Determining the Regulation of Drug Efflux/Drug Clearance

Methods have been described for the identification and determination of agonists and non-agonists (including antagonists) of xenobiotic metabolism and drug-metabolizing enzyme activity by molecules, such as drug leads. All of the screening methods and concepts described above are equally applicable for determining the regulation of drug efflux or drug clearance and are intended to be applicable to such. The discussion below is illustrative of the methods, but it should be understood that the discussion and concepts above of agonists and non-agonists (including antagonists) and the effect on a nuclear receptor that regulates cytochrome P450 expression (and the resulting effect on xenobiotic metabolism) is transferrable to the effect on a receptor that regulates drug transport proteins (effecting on drug efflux or clearance).

The concepts of drug efflux/drug clearance are described in Schuetz & Strom, Nat. Med. 7:536-537 and Synold et al, Nat Med. 7:584-590. These references are incorporated by reference in their entireties.

For example, the present invention can be used to identify an agonist of drug clearance by screening a molecule for its ability to modify the stability of a receptor regulating expression of a drug transport protein and to further modify the stability of the receptor when in the presence of one or more co-activators. A molecule that modifies the stability of the receptor and further modifies the stability of the receptor when in the presence of a co-activator can be identified as an agonist of drug clearance.

Also, an agonist of drug clearance can be determined by screening a molecule for its ability to shift the thermal unfolding curve of a receptor regulating expression of a drug transport protein and to further shift the thermal unfolding curve of the receptor when in the presence of one or more co-activators. A molecule that shifts the thermal unfolding curve of the receptor and further shifts the thermal unfolding curve of the receptor when in the presence of a co-activator can be identified as an agonist of drug clearance.

The effect on the activity of drug efflux of a drug lead can be determined by providing a drug lead that modifies the stability of a receptor regulating expression of a drug transport protein and screening the drug lead for its ability to further modify the stability of the receptor in the presence of one or more co-regulators. Whether there is a further modification of stability of the receptor in the presence of the drug lead and a co-regulator is an indication whether the drug lead increases the activity of drug efflux.

Also, the effect on the activity of drug efflux of a drug lead can be determined by providing a drug lead that shifts the thermal unfolding curve of a receptor regulating expression of a drug trrnsport protein and screening the drug lead for its ability to, further shift the thermal unfolding curve of the receptor in the presence of one or more co-regulators. Whether there is a further shift in the thermal unfolding curve of the receptor in the presence of the drug lead and a co-regulator is an indication as to whether the drug lead increases the activity of drug efflux.

Also, the effect of a molecule on xenobiotic metabolism and/or drug clearance may be determined using the present invention. It has been reported that the SXR receptor regulates drug catabolism by regulating cytochrome P450 expression and drug transport proteins. The inventive method comprises screening a molecule for its ability to modity the stability of the SXR receptor and to further modify the stability of the receptor when in the presence of one or more co-regulators. A further modification of stability of the receptor in the presence of the molecule and a co-regulator of said one or more co-regulators indicates whether the molecule is an agonist or an antagonist of xenobiotic metabolism and/or drug clearance.

The effect of a molecule on xenobiotic metabolism and/or drug clearance may also be determined by screening a molecule for its ability to shift the thermal unfolding curve of the SXR receptor and to further shift the thermal unfolding curve of the receptor when in the presence of one or more co-regulators. A further shift of the thermal unfolding curve of the receptor in the presence of the molecule and a co-regulator of indicates whether the molecule is an agonist or an antagonist of xenobiotic metabolism and/or drug clearance.

Although the ligand binding domain of receptors regulating cytochrome P450 expression and drug transport proteins, ligands and co-regulators that interact with this domain is described, the invention can be extended to the full length protein, in the presence of additional regulators and finally in the presence of DNA.

Also, these studies are not limited for protein-protein interactions but also can be used for protein-peptide interactions where the peptides represent short linear sequences representing protein domains that interact preferentially with the protein of interest.

Having now generally described the invention, the same will become more readily understood by reference to the following specific examples which are included herein for purposes of illustration only and are not intended to be limiting unless otherwise specified.

### EXAMPLES

Experimental Results for Nuclear Receptors

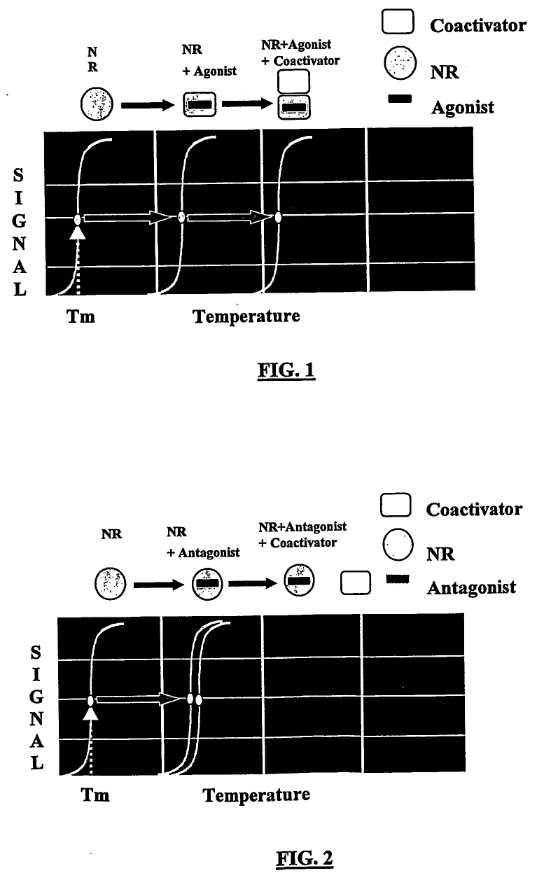

The experimental results expected for an agonist response vs. an antagonist response in the presence of a co-activator is shown in FIGS. 1 and 2. In the case of an agonist ligand and in the presence of co-activator protein/peptide the prediction is an increase in the stability of the receptor (FIG. 1), while for an antagonist no additional stabilization will be observed (FIG. 2).

### Example 1

Table 1 is a summary of the data obtained for ER-α and ER-β for the study of a panel of four known agonist and three known antagonists in the presence of a co-activator protein SRC-3; in the presence of two co-activator peptides SRC1-NR2 and SRC3-NR2 derived from the sequence of the co-activators SRC-1 and SRC-3; and in the presence of the co-repressor peptide NCoR-1 derived from the co-repressor NCoR-1.

The concentration of ER-α and ER-β in all of the experiments was 8 μM, the ligand concentration was 20 μM, SRC-3 was 11 μM, and the co-regulator peptides SRC1-NR2, SRC3-NR2, and NCoR-1 was at 100 μM. The experiments were performed in 25 mM phosphate pH 8.0, 200 mM NaCl, 10% glycerol and in the presence of 25 μM dapoxyl sulfonamide dye (available from Molecular Probes, Inc., Eugene, Oreg.).

A 2 μM ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus (see U.S. Pat. Nos. 6,020,141; 6,036,920; 6,291,191; 6,268,218; 6,232,085; 6,268,158; 6,214,293; 6,291,192; and 6,303,322) and analyzed using non-linear least squares fitting software. The results listed below are the average of four experiments. The values for the co-regulators represent a change in Tₘ stabilization from the receptor-ligand ΔTₘ values.

TABLE 1
Observed ΔTₘ Stabilization of ER-α in the Presence of Ligands and the
Co-activator Proteins SRC-1 and SRC-3

| | SRC-3 | SRC1- | NR2 | SRC3-NR2 | NCoR1-NR1 |
|---|---|---|---|---|---|
| ER-α | 0.0 | 1.5 | 0.8 | 0.9 | 0.0 |
| Estradiol | 14.8 | 3.8 | 4.9 | 4.3 | 0.0 |
| Estrone | 7.7 | 3.5 | 3.0 | 2.3 | −0.3 |
| 17-α-ethylene-E2 | 15.5 | 4.5 | 4.5 | 3.9 | −0.1 |
| 2-methoxy-E2 | 3.5 | 5.3 | 5.5 | 4.3 | −0.7 |
| tamoxifen | 8.5 | 1.1 | −0.5 | 0.0 | 0.1 |
| 4-OH-tamoxifen | 17.7 | 0.2 | 0.2 | 0.7 | 0.1 |
| ICI-182780 | 13.9 | 0.5 | 0.2 | 0.2 | −0.6 |
| ER-β | 0.0 | 0.9 | 0.7 | 0.9 | −0.4 |
| Estradiol | 17.5 | 1.7 | 3.4 | 3.5 | 0.0 |
| Estrone | 11.3 | 1.6 | 3.8 | 3.7 | −0.3 |
| 17-α-ethylene-E2 | 15.3 | 2.6 | 2.6 | 2.6 | −0.7 |
| 2-methoxy-E2 | 2.9 | 2.5 | 4.4 | 4.4 | −0.7 |
| tamoxifen | 9.8 | 1.3 | 0.2 | 0.4 | 0.4 |
| 4-OH-tamoxifen | 18.2 | 1.2 | 0.2 | 0.8 | 0.3 |
| ICI-182780 | 16.7 | 0.9 | 0.4 | 0.6 | 0.3 |

From the above results, from counter-screening in the presence of co-activator protein/peptide in the presence of the estrogen-like compounds, an additional stabilization was observed for both receptors. Thus, these compounds act like agonists in agreement with literature. The tamoxifen and ICI compound are known antagonists and they have no ability to recruit co-activators. This is also in agreement with the literature.

### Example 2

Table 2 is a summary of the data obtained for SXR for the study of a panel of known steroid and drug ligands; in the presence of the co-activator peptide SRC1-NR2 derived from the sequence of the co-activators SRC-1; and in the presence of the co-repressor peptide NCoR-1 derived from the co-repressor NCoR-1.

The concentration of SXR in all of the experiments was 6 μM, the ligand concentration was 50 μM, and the co-regulator peptides SRC1-NR2, and NCoR-1 was at 100 μM. The experiments were performed in 25 mM HEPES pH 7.9, 200 mM NaCl, 5% glycerol and in the presence of 25 μM dapoxyl sulfonamide dye (available from Molecular Probes, Inc., Eugene, Oreg.).

A 2 μL ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus (see U.S. Pat. Nos. 6,020,141; 6,036,920; 6,291,191; 6,268,218; 6,232,085; 6,268,158; 6,214,293; 6,291,192; and 6,303,322) and analyzed using non-linear least squares fitting software. The results listed below are the average of four experiments. The values for the co-regulators represent a change in Tₘ stabilization from the receptor-ligand ΔTₘ values. See, e.g., Hugh D. Young, Statistical Treatment of Experimental Data (1962); White, Robert S., Statistics (1989); Pitman, Jim, Probability (1993); and Brown, Byron W., Statistics, A Biomedical Introduction (1977).

TABLE 2
Observed Stabilization of SXR in the Presence of Ligands and the
Co-activator Peptide SRC1-NR2 and Co-Repressor Peptide NCoR-1

| | | ΔΔTm | ΔΔTm NcoR1- |
|---|---|---|---|
| Ligand | ΔTm ligand | SRC1-NR2 | NR2 |
| Ligand free | — | 2.9 | 6.7 |
| Taxol | 1.3 | 2.5 | 4.7 |
| Cortisone | 1.8 | 2.9 | 3.6 |
| 4-androstene | 4.3 | 2.9 | 3.6 |
| Hydrocortisone | 1.6 | 3.3 | 3.7 |
| Androsterone | 3.3 | 3.5 | 3.8 |
| 17-α-hydroxyprogesterone | 1 | 3.3 | 3.5 |
| Clofibrate | 1.3 | 3.3 | 3.4 |
| Estradiol | 2.3 | 3.9 | 2.3 |
| Lithocholic acid | 2.4 | 2.9 | 1.4 |
| Lovastatin | 6.3 | 3.0 | 1.2 |
| Corticosterone | 5.0 | 2.7 | 0.5 |
| 11-α-hydroxy-progesterone | 6.3 | 4.6 | 1.0 |

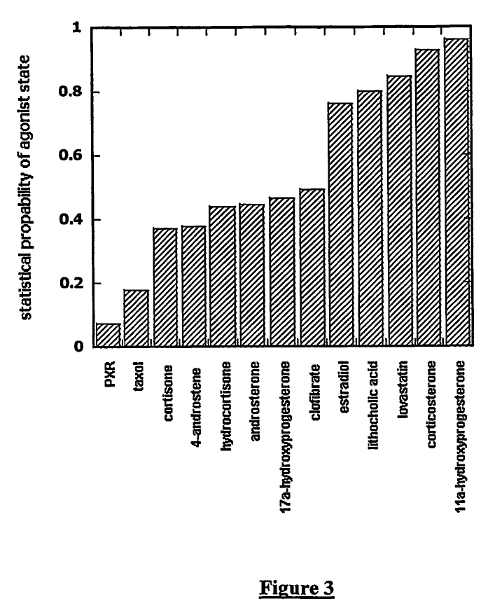

FIG. 3 illustrates the calculated statistical probability for the receptor to be in an activated conformation computed from the affinities derived from the observed changes in stability of the receptor in the presence of the co-regulator peptide for a given ligand (ΔΔTₘ values are from Table 2).

From Table 2 and FIG. 3 we can conclude the following:

a) All xenobiotic ligands and steroids affect differentially recruitment of co-activator and co-repressor peptides.

b) All ligands recruit co-activator peptide more efficiently with the exception of taxol (ΔΔTm values for SRC1-NR2 from Table 2).

c) No compound recruits co-repressor peptide more efficiently than the ligand free receptor (ΔΔTm values for NcoR1-NR1 from Table 2).

TABLE 3
Calculated Affinities for SXR ligands and Relationship of Statistical
Probability of Agonist State to known Pharmacological Response.

| | Ka ligand | Probability | fold | EC₅₀ |
|---|---|---|---|---|
| Ligand | (nM) | Agonist | activation | (nM) |
| Taxol | 23000 | 0.18 | | 5000 |
| Cortisone | 13000 | 0.37 | No- | |
| | | | activation | |
| 4-androstene | 2000 | 0.38 | | |
| Hydrocortisone | 17000 | 0.44 | | |
| Androsterone | 4100 | 0.45 | | |
| 17-α-hydroxyprogesterone | 33000 | 0.47 | 1.5 fold | |
| Clofibrate | 23000 | 0.49 | | |
| Estradiol | 8700 | 0.76 | 3-fold | |
| Lithocholic acid | 8300 | 0.8 | | 9000 |
| Lovastatin | 550 | 0.85 | 5-fold | |
| Corticosterone | 1320 | 0.93 | 12 fold | |
| 11-α-hydroxy-progesterone | 550 | 0.96 | | |

Data from columns 4 and 5 have been obtained from Ekins & Erickson, Drug Metabolism and Disposition, 30, 96-99, 2003 and references therein.

From the data in Table 3 we can conclude the following:

a) All ligands tested will perturb basal biological state of SXR.

b) Probability of agonist state is correlated to the reported fold activation for a subset of compounds from literature.

c) Biological effect of these compounds on the induction of P450's will depend both on the affinity of the compound for the receptor and the ability of the receptor to distinguish between co-activators and co-repressors.

Therefore compounds that have a high affinity for the receptor (binding affinities of about 5 μm and lower) and high probability of agonist state (statistical probability of agonist state is about 0.8 to about 1.0) will be strong inducers of P450 expression, intermediate agonist probability (statistical probability of agonist state of about 0.4 to about 0.8) will appear as weak inducers of P450 expression, and those with a low agonist probability (statistical probability of agonist state of about 0.0 to about 0.4) will appear inactive. On the other hand weak interacting compounds (binding affinities of about 5 μM and higher) with a high or intermediate probability of an agonist state (statistical probability of agonist state of about 0.4 to about 1.0) will appear as weak inducers for P450 expression, and those with a low probability of agonist state (statistical probability of agonist state of about 0.0 to about 0.4) will appear as inactive.

The results shown above in Example 1 illustrate how the present invention may be used to identify agonists and antagonists of nuclear receptors ER-α and ER-β. The results from example 2 illustrate how the present invention can be used to identify molecules such as drug candidates or leads for their effect on a drug-metabolizing enzyme/xenobiotic metabolism or drug clearance by screening ligands for their ability to shift the thermal unfolding curve of the SXR. In the same fashion, the present invention can be used to identify molecules such as drug candidates or leads for their effect on a drug-metabolizing enzyme/xenobiotic metabolism or drug clearance by screening ligands for their ability to shift the thermal unfolding curve of the AhtXRE, CAR and PPAR-α receptor, nuclear receptors that regulates cytochrome P450 expression or drug transport proteins, respectively.

The thermal shift assay described in U.S. Pat. Nos. 6,020,141; 6,036,920; 6,291,191; 6,268,218; 6,232,085; 6,268,158; 6,214,293; 6,291,192; and 6,303,322, (Thermofluor®) can measure the change in melting temperature that accompanies the binding of a steroid or xenobiotic to a nuclear receptor involved in the regulation of cytochrome P450 expression or drug transport proteins. The thermal shift assay can then determine the added effect of binding of the binding domain of a coactivator or corepressor protein to the liganded nuclear receptor.

The effect of a xenobiotic on the system can be accomplished as follows: First, a series of test reagents can be screened for the ability to bind to and change the melting temperature of a nuclear receptor that regulates cytochrome P450 expression or drug transport proteins. Next, the ability of the binding domains of coactivator and corepressor proteins to further shift the melting temperature of the complex can be tested (peptide fragments can be substituted). Based on the pattern of effects, one can predict changes in the expression level of drug metabolizing enzymes or drug efflux.

If a coactivator protein adds to the thermal stability of the complex then the xenobiotic is predicted to be an agonist that will stimulate the expression of P450 enzyme or drug trasport proteins, respectively. If the corepressor domain binds to the binary complex then the xenobiotic is predicted to be an antagonist of P450 expression or drug transport proteins, respectively. If the xenobiotic does not bind to the nuclear receptor then the compound will be predicted not to affect the expression of a P450 enzyme or drug transport proteins, respectively. Therefore, the thermal shift assay can be used to predict changes in the levels of drug metabolizing enzymes or clearance of the drug caused by a drug candidate or xenobiotic.

Schuetz & Strom, Nat. Med. 7:536-537 and Synold et al, Nat. Med. 7:584-590, report that SXR (also known as PXR) regulates xenobiotic metabolism, and also regulates drug efflux by activating expression of the gene MDR1, which encodes the protein P-glycoprotein.

By the methods described above, the SXR, Ah/XRE, CAR and PPAR-α receptor can be screeened to determine the effect of xenobiotics on the thermal unfolding curve of the receptor in the presence of co-activator and co-repressor proteins to determine what effect the xenobiotic has on xenobiotic metabolism and/or drug efflux.

All publications and patents mentioned hereinabove are hereby incorporated in their entireties by reference. Also, the use of the term “a” in the application is intended to include both the singular and plural, ie. one or more.

While the foregoing invention has been described in some detail for the purposes of clarity and understanding, it will be appreciated by one skilled in the art from a reading of this disclosure that various changes in form and detail can be made without departing from the true scope of the invention and appended claims.

1-9. (canceled)

10. A method of determining the effect of a drug lead on the activity of a drug-metabolizing enzyme comprising:
(a) providing a-drug lead that shifts the thermal unfolding curve of a receptor regulating cytochrome P450 expression; and
(b) screening the drug lead for its ability to further shift the thermal unfolding curve of the receptor in the presence of one or more co-regulators; wherein a further shift in the thermal unfolding curve of the receptor in the presence of the drug lead and a co-regulator of said one or more co-regulators indicates whether the drug lead increases the activity of a drug-metabolizing enzyme.

11. The method of claim 10, wherein providing a drug lead that shifts the thermal unfolding curve of the receptor comprises screening one or more of a multiplicity of different molecules for their ability to shift the thermal unfolding curve of the receptor.

12. The method of claim 11, wherein said screening of one or more of a multiplicity of different molecules comprises:
(a) contacting said receptor regulating cytochrome P450 and one or more molecules in each of a multiplicity of containers;
(b) heating said receptor in each of said multiplicity of containers to cause said receptor to unfold;
(c) measuring in each of said containers a physical change associated with the thermal unfolding of said receptor;
(d) generating a thermal unfolding curve for said receptor for each of said containers;
(e) comparing each of said thermal unfolding curves in step (d) to:
(i) each of the other thermal unfolding curves; and/or
(ii) the thermal unfolding curve for said target molecule in the absence of any of said multiplicity of molecules; and
(f) determining whether any of said multiplicity of molecules shifts the thermal unfolding curvte of said receptor.

13. The method of claim 10, wherein said screening step further comprises:
(a) contacting said drug lead and said receptor regulating cytochrome P450 expression with one or more of said co-regulators in each of a multiplicity of containers;
(b) heating said receptor in each of said multiplicity of containers to cause said receptor to unfold;
(c) measuring in each of said containers a physical change associated with the thermmal unfolding of said receptor;
(d) generating a thermal unfolding curve for said receptor for each of said containers;
(e) comparing each of said thermal unfolding curves in step (d) to:
(i) each of the other thermal unfolding curves; and/or
(ii) the thermal unfolding curve for said receptor in the absence of (1) said drug lead and/or (2) said co-regulators; and
(f) determining whether said drug lead further shifts the thermal unfolding curve of said receptor.

14. The method of claim 10 wherein the one or more co-regulators includes a co-activator and/or co-repressor.

15. The method according to claim 10, wherein the molecule-fiurtier modifies the stability of the receptor in the presence of a co-activator, thereby identifying the ligand as an agonist of the receptor when in the presence of the co-activator.

16. The method according to claim 15, wherein the agonist is a partial agonist.

17. The method according to claim 10, wherein the molecule further modifies the stability of the receptor in the presence of a co-repressor, thereby identifying the ligand as a non-agonist of the receptor when in the presence of the co-activator.

18. The method according to claim 17, wherein the non-agonist is a partial agonist.

19-21. (canceled)

22. A method of identifying an agonist of xenobibtic metabolism comprising screening a molecule for its ability to shift the thermal unfolding curve of a receptor regulating cytochrome P450 expression and to further shift the thermal unfolding curve of said receptor when in the presence of one or more co-activators; wherein a molecule that shifts the thermal unfolding curve of said receptor and further shifts the thermal unfolding curve of said receptor when in the presence of a co-activator is identified as an agonimst of xenobiotic metabolism.

23. The method of claim 22, wherein said screening step further comprises:
(a): contacting said molecule and said receptor regulating cytochrome P450 expression with one or more of said co-activators in each of a multiplicity of containers;
(b) heating said receptor in each of said multiplicity of containers to cause said receptor to unfold;
(c) measuring in each of said containers a physical change associated with the thermal unfolding of said receptor;
(d) generating a thermal unfolding curve for said receptor for each of said containers;
(e) comparing each of said thermal ufolding curves in step (d) to:
(i) each of the other thermal unfolding curves; and/or
(ii) the thermal unfolding curve for said receptor in the absence of (1) said molecule and/or (2) said co-activators; and
(f) determining whether said molecule further modifies the stability of said receptor, wherein a further modification in stability is indicated by a further change in said uhfolding curve.

24. A method according to claim 22, wherein the agonist is a partial agonist.

25-26. (canceled)

27. A method of identifying a non-agonist of xenobiotic metabolism comprising screening a molecule for its ability to shift the thermal unfolding curve of a receptort regulating cytochrome P450,expression, wherein a molecule that does not shift the thermal unfolding curve of said receptor is identified as a non-agonist of xenobiotic metabolism.

28. The method of claim 27, wherein said screenig step comprises:
(a) contacting said receptor regulating cytochrome P450 and one or more molecules in each of a multiplicity of containers;
(b) heating said receptor in each of said multiplicity of containers to cause said receptor to unfold;
(c) measuring in each of said containers a physical change associated with the thermal unfolding of said receptor;
(d) generating a thermal unfolding curve for said receptor for each of said containers;
(e) comparing each of said thermal unfolding curves in step (d) to:
(i) each of the other thermal unfolding curves; and/or
(ii) the thermal unfolding curve for said target molecule in the absence of any of said multiplicity of molecules; and
(f) determining whether said molecule modifies the thermal unfolding curve of said receptor.

29-60. (canceled)

61. A method according to claim 10, wherein the co-regulator is a co-activator and/or a co-repressor.

62. A method according to claim 10, wherein an agonist for a co-regulator dependent receptor is a strong inducer.

63. A method according to claim 62, wherein the strong inducer is 11-α-hydroxyprogesterone.

64. A method according to claim 10, wherein the strong inducer has a binding affinity of less than about 5 μM and a statistical probability of agonist state of about 0.8 to about 1.0.

65. A method according to claim 10, wherein a partial agonist of a co-regulator dependent receptor is a weak inducer.

66. A method according to claim 65, wherein the weak inducer has a binding affinity of less than about 5 μM and a statistical probability of agonist state of about 0.4 to about 0.8.

67. A method according to claim 65, wherein the weak inducer has a binding affinity of at least about 5 μM and a statistical probability of agonist state of about 0.4 to about 1.0.

68. A method according to claim 10, wherein an antagonist of a co-regulator dependent receptor is a non-inducer.

69. A method according to claim 68, wherein the non-inducer has a binding affinity of less than about 5 μM and a statistical probability of agonist state of less than about 0.4.

70. A method according to claim 65, wherein the non-inducer has a binding affinity of at least about 5 μM and a statistical probability of agonist state of less than about 0.4.
